(19) United States
(12) Patent Application Publication
(10) Pub. No.: US 2025/0231115 A1
(43) Pub. Date: Jul. 17, 2025

(54) System for Measuring Microplastics in an Aquatic Environment

(71) Applicant: Ely Oser, Brooklyn, NY (US)

(72) Inventor: Ely Oser, Brooklyn, NY (US)

(21) Appl. No.: 19/030,690

(22) Filed: Jan. 17, 2025

Related U.S. Application Data
(60) Provisional application No. 63/621,938, filed on Jan. 17, 2024.

(51) Int. Cl. G01N 21/88; G01N 33/44; G01N 35/00

(52) U.S. Cl. CPC G01N 21/8806; G01N 33/442; G01N 2035/00306; G01N 2201/062; G01N 2223/101; G01N 2223/623

(57) ABSTRACT

A system for measuring microplastics in an aquatic environment is a system that provides practical and accurate means of assessing the presence of plastics in a body of water. The system may include an electronics housing, Electromagnetic (EM) radiation emitters, a photoresistor, a controller, a power source, and a fluid conduit. The electronics housing is a portable hermetic structure that protects the electronic and electrical components from water damage. The EM radiation emitters emit Ultraviolet (UV) light that is absorbed by the microplastics in the sample water being analyzed. The photoresistor detects the light emitted by the microplastics in the sample water after absorbing the UV light. The controller processes the sensor signals from the at least one photoresistor to generate analysis data. The power source provides the energy necessary for the operation of the system. The fluid conduit enables the controlled flow of the sample water for analysis.

The current application claims a priority to the U.S. provisional patent application Ser. No. 63/621,938 filed on Jan. 17, 2024.

### FIELD OF THE INVENTION

The present invention relates generally to aquatic systems and water pollution assessment systems. More specifically, the present invention discloses a system that detects microplastic levels in aquatic environments by measuring the microplastic fluorescence in terms of light intensity.

### BACKGROUND OF THE INVENTION

Due to increasing pollution in water, different analysis systems and methods are available to measure the presence of pollutants in water, especially plastics. Traditional systems and methods for measuring the presence of plastics in water are not accurate because microplastics in large bodies of water are unevenly distributed due to variations in size, density, shape, and deep-water currents. This results in random location sampling not producing an accurate estimate of varying plastic concentrations. Additionally, current systems and methods lack real-time data, requiring laboratory analysis and delayed feedback. Novel means are required to properly assess the presence of plastics in water. For example, when plastics are subjected to Ultraviolet (UV) light in dark environments, plastics exhibit fluorescence, a process where plastics absorb light, or other Electromagnetic (EM) radiation, and light is emitted. This phenomenon can be used to measure the amount of plastics in a water sample, as the fluorescence intensity is generally proportional to the concentration of plastics in the water sample. However, no practical system has been developed that utilizes this phenomenon to assess the presence of plastics in a body of water.

Therefore, an objective of the present invention is to provide a system for measuring microplastics in an aquatic environment that is based on the fluorescence properties of plastics. The system of the present invention measures the intensity of fluorescence light emitted by plastics in dark environments. This way, the present invention effectively overcomes the limitations of the traditional detection methods, sieving and sampling water to determine the quantity of microplastics relative to the volume of water. Additional features and benefits of the present invention are further discussed in the sections below.

### SUMMARY OF THE INVENTION

The present invention discloses a system for measuring microplastics in an aquatic environment. The present invention offers real-time results, enabling immediate measurement and continuous monitoring of microplastic concentration in a body of water. To do so, the present invention discloses a sensor system that can be deployed to the target body of water for analysis. Additionally, the sensor system is cheap, fast, and energy-efficient; the sensor circuitry of the system is basic and uses standard industry components. The sensor system is preferably provided as a versatile structure that can be easily attached to moving marine vehicles including, but not limited to, maritime ships, submarines, and other maritime vessels. This allows measurements to be taken while in motion, and in real-time while traveling along the target body of water. Further, the present invention can be implemented within a fluid conduit that reroutes sample water from the target body of water without external light interference. This allows for accurate assessment of the presence of plastics on the target body of water. Thus, the present invention can provide valuable real-time insights into the aggregational patterns and higher concentration areas of microplastics in different bodies of water.

### BRIEF DESCRIPTION OF THE DRAWINGS

FIG. 1 is a top-front perspective view of the electronics housing of the system of the present invention.

FIG. 2 is a bottom-front perspective view of the electronics housing of the system of the present invention.

FIG. 3 is a top view of the electronics housing of the system of the present invention.

FIG. 4 is a front view of the electronics housing of the system of the present invention.

FIG. 5 is a cross-sectional view of the electronics housing taken along line 5-5 in FIG. 4.

FIG. 6 is a top-front perspective view of the system of the present invention.

FIG. 7 is a bottom-rear perspective view of the system of the present invention.

FIG. 8 is a front view of the system of the present invention.

FIG. 9 is a cross-sectional view of the system of the present invention taken along line 9-9 in FIG. 8.

FIG. 10 is a box diagram showing the electronic connections and the electrical connections of the present invention, wherein the electronic connections are shown in dashed lines, wherein the electrical connections are shown in solid lines, and wherein the controller and the power source are shown implemented inside the electronics housing.

FIG. 11 is a box diagram showing the electronic connections and the electrical connections of the present invention, wherein the electronic connections are shown in dashed lines, wherein the electrical connections are shown in solid lines, and wherein the controller and the power source are shown implemented external to the electronics housing.

### DETAILED DESCRIPTION OF THE INVENTION

All illustrations of the drawings are for the purpose of describing selected versions of the present invention and are not intended to limit the scope of the present invention.

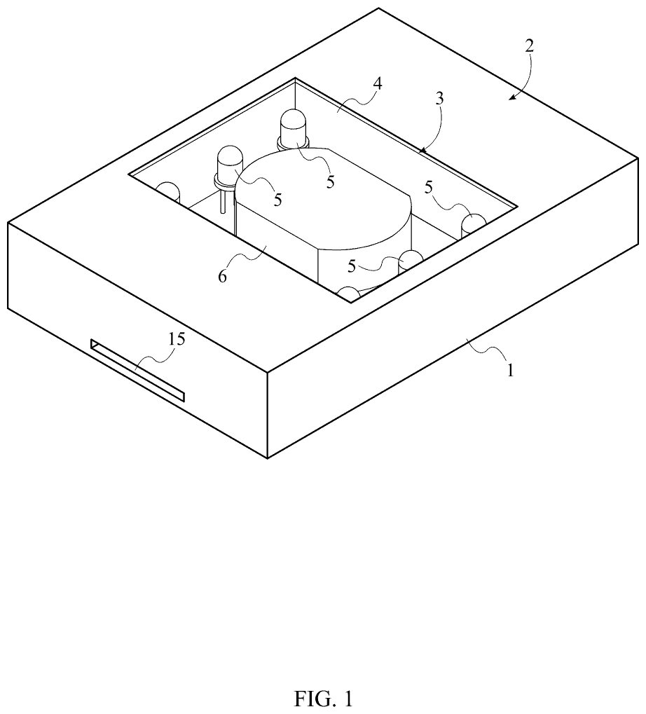

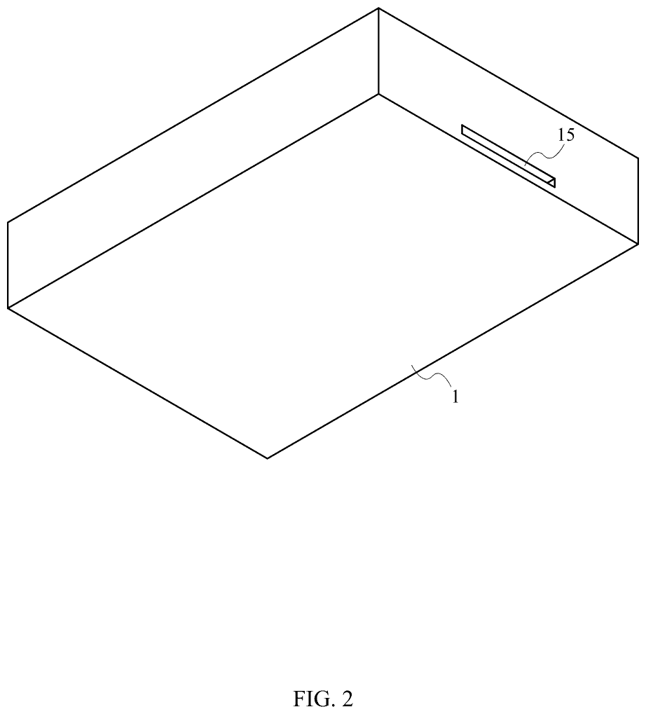

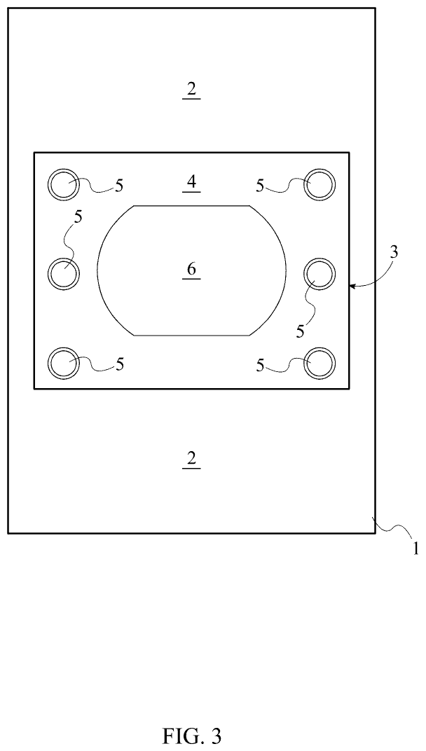

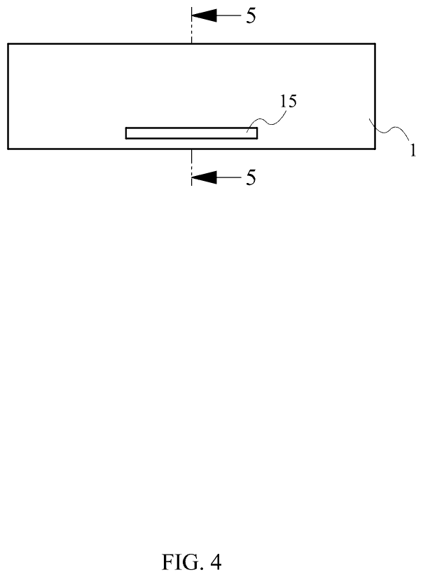

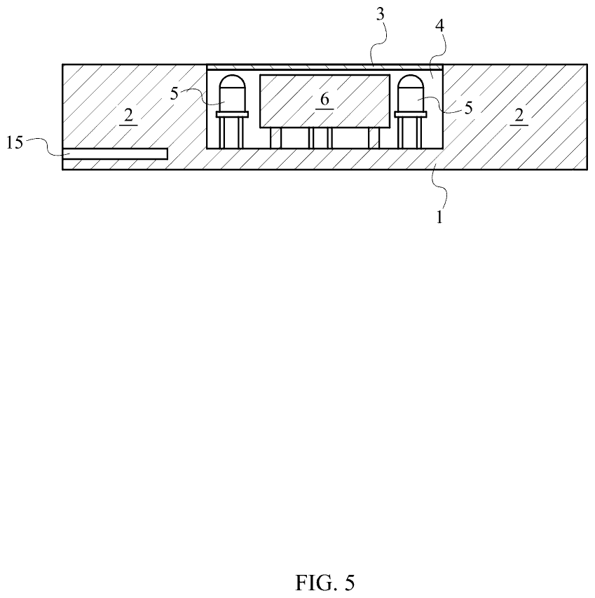

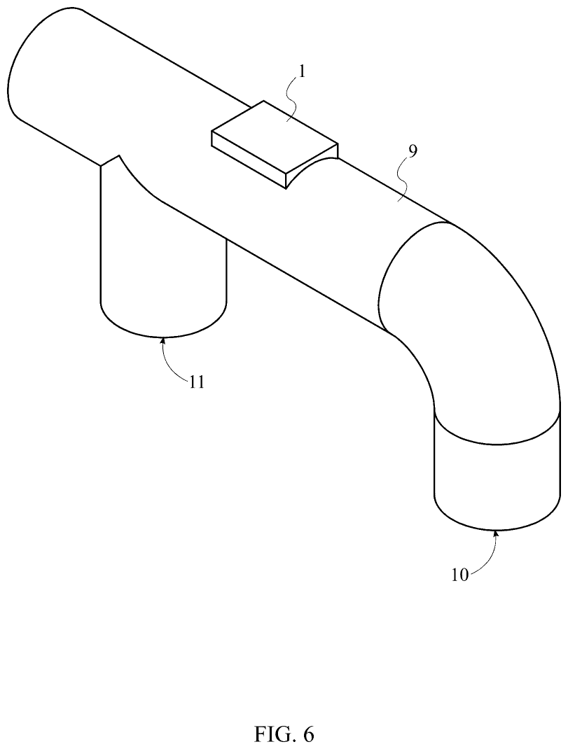

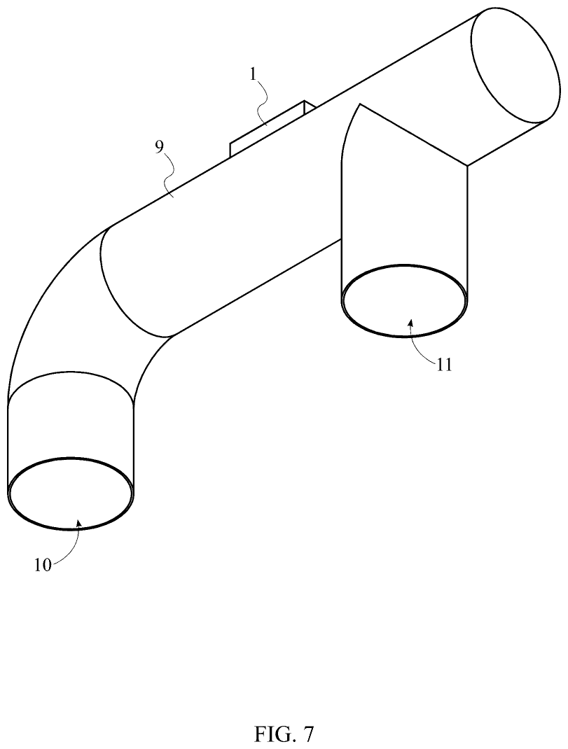

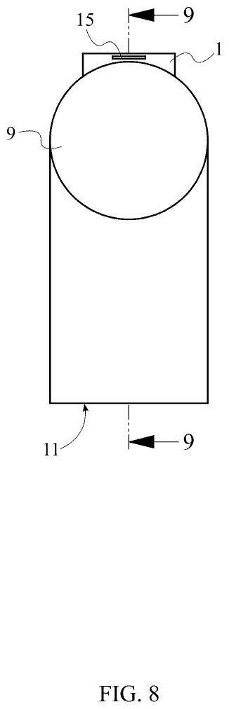

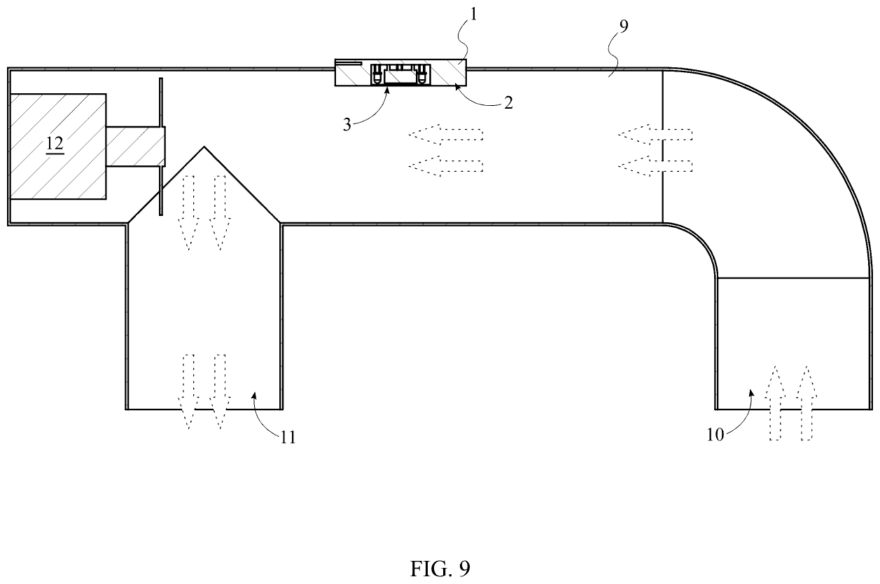

The present invention discloses a system for measuring microplastics in an aquatic environment. The present invention provides practical and accurate means of assessing the presence of plastics in a body of water. As can be seen in FIGS. 1 through 11, the present invention comprises an electronics housing 1, a plurality of electromagnetic (EM) radiation emitters 5, at least one photoresistor 6, a controller 7, a power source 8, and a fluid conduit 9. The electronics housing 1 is a portable hermetic structure that protects the electronic and electrical components from water damage. The plurality of EM radiation emitters 5 emits Ultraviolet (UV) light that is absorbed by the microplastics in the sample water being analyzed. The at least one photoresistor 6 detect the light emitted by the microplastics in the sample water after absorbing the UV light. The controller 7 processes the sensor signals from the at least one photoresistor 6 to generate the corresponding analysis data. The power source 8 provides the electrical energy necessary for the operation of the system. The fluid conduit 9 enables the controlled flow of the sample water towards the system for analysis.

The general configuration of the aforementioned components enables the capture of valuable insights into the aggregation patterns and higher concentration areas of microplastics in a target body of water. As can be seen in FIGS. 1 through 9, the electronics housing 1 is preferably a small hollow structure large enough to accommodate the plurality of EM radiation emitters 5 and the at least one photoresistor 6. The electronics housing 1 helps guide the EM radiation from the plurality of EM radiation emitters 5 towards the sample water flowing through the fluid conduit 9. For example, the plurality of EM radiation emitters 5 is a plurality of UV light emitting diodes (LEDs) shine UV light on the possible microplastics carried in the sample water. In addition, the electronics housing 1 is designed to enable only the light emitted by the microplastics in the sample water to reach the at least one photoresistor 6.

In general, the electronics housing 1 comprises an opaque housing portion 2 and a transparent housing portion 3, as can be seen in FIGS. 1 through 9. The opaque housing portion 2 prevents external light from reaching the at least one photoresistor 6. On the other hand, the transparent housing portion 3 enables only the light emitted by the microplastics in the sample water to reach the at least one photoresistor 6. For example, the electronics housing 1 can be a thin rectangular structure, with the opaque housing portion 2 corresponding to the overall perimeter of the rectangular structure, and the transparent housing portion 3 corresponding to a central section of the rectangular structure where the plurality of EM radiation emitters 5 and the at least one photoresistor 6 are positioned. Furthermore, the fluid conduit 9 can be made from an opaque material to prevent external light from the surroundings from corrupting the collected data if the external light were to reach the at least one photoresistor 6.

In general, the present invention can be arranged as follows: the opaque housing portion 2 is perimetrically positioned around the transparent housing portion 3 so that the opaque housing portion 2 surrounds the transparent housing portion 3, as can be seen in FIGS. 1 through 11. The opaque housing portion 2 is also mounted within the fluid conduit 9 to secure the electronics housing 1 inside the fluid conduit 9. Further, the transparent housing portion 3 is oriented towards an interior of the fluid conduit 9 so that the plurality of EM radiation emitters 5 and the at least one photoresistor 6 are exposed to the sample water flowing through the fluid conduit 9. Moreover, the plurality of electromagnetic radiation emitters and the at least one photoresistor 6 are mounted within the electronics housing 1 to secure the plurality of EM radiation emitters 5 and the at least one photoresistor 6 inside the electronics housing 1. The plurality of electromagnetic radiation emitters and the at least one photoresistor 6 are positioned adjacent to the transparent housing portion 3. This way, the emitted EM radiation is directed to the sample water containing possible microplastics, and the light emitted by the radiated microplastics can reach the at least one photoresistor 6.

As can be seen in FIGS. 1 through 11, the plurality of electromagnetic radiation emitters and the at least one photoresistor 6 are electronically connected to the controller 7. The controller 7 can be a micro computing device that can be implemented on the electronics housing 1. Alternatively, the controller 7 can be a portable computing device that can be selectively connected to the system when analyzing the target body of water. This way, the controller 7 can oversee the operation of the plurality of EM radiation emitters 5 by relaying the appropriate command signals to the plurality of EM radiation emitters 5. Further, the sensor signals from the at least one photoresistor 6 can be relayed to the controller 7 for processing so that the corresponding analysis data can be generated by the controller 7. Moreover, the plurality of light sources, the at least one photoresistor 6, and the controller 7 are electrically connected to the power source 8. Like the controller 7, the power source 8 can be a portable power storage device, like one or more rechargeable or replaceable batteries, that is implemented in the electronics housing 1, or an external power source 8 that is selectively connected to the system when analyzing the target body of water.

As previously discussed, the plurality of EM radiation emitters 5 and the at least one photoresistor 6 are positioned adjacent to the transparent housing portion 3. As can be seen in FIGS. 1 through 5, to accommodate the plurality of EM radiation emitters 5 and the at least one photoresistor 6, the electronics housing 1 may further comprise a housing slot 4. The housing slot 4 corresponds to a space on the electronics housing 1 that positions the plurality of EM radiation emitters 5 and the at least one photoresistor 6. In addition, the transparent housing portion 3 is a transparent slot cover that fits on the opening of the housing slot 4. For example, a piece of acrylic or similar transparent material can be utilized for the transparent slot cover. To implement the housing slot 4, the housing slot 4 traverses through the opaque housing portion 2 and into the electronics housing 1 to form a space large enough to accommodate the plurality of EM radiation emitters 5 and the at least one photoresistor 6. Further, the transparent slot cover is hermetically mounted into the housing slot 4 to prevent water damage to the electronics housed within.

Furthermore, the plurality of EM radiation emitters 5 and the at least one photoresistor 6 can be arranged into different configurations inside the housing slot 4 for the most efficient and accurate analysis of the sample water flowing through the fluid conduit 9. In the preferred embodiment, as can be seen in FIGS. 1 through 5, the plurality of EM radiation emitters 5 is distributed around the at least one photoresistor 6 inside the housing slot 4. In other words, the plurality of EM radiation emitters 5 is arranged in such a way that the EM radiation emitters surround the at least one photoresistor 6. In other embodiments, different arrangements can be implemented according to the design of the electronics housing 1.

Furthermore, the at least one photoresistor 6 must be calibrated such that the at least one photoresistor 6 gives a value of weight (of plastics) to volume (of water) ratio, in grams per cubic decimeter. The at least one photoresistor 6 can be calibrated using standard solutions of microplastics. For example, solutions can be created by first weighing the plastic and then breaking the plastic down into water-based solutions to obtain a weight to volume ratio. The solution is then passed through the at least one photoresistor 6 to obtain an intensity reading. With multiple intensity readings corresponding to different microplastic concentrations, a regression model can be generated enabling the prediction of future microplastic concentrations solely based on intensity readings.

Further, the at least one photoresistor 6 is a variable resistor whose resistance changes depending on the amount of light it is exposed to. To determine the concentration of microplastics, the intensity value across the at least one photoresistor 6 is examined through calibration. However, this does not give an exact value on the concentration of microplastics in a given volume. As discussed previously, a scale must be established between the intensity reading across the at least one photoresistor 6 and a mass-to-volume ratio to determine the mass of microplastics in a given volume of water.

However, this method does not yield an exact value for the microplastic concentration in a given volume. Only 40% of plastics fluoresce under UV light, indicating that only 40% of plastic is detectable. Therefore, the obtained value must be multiplied by a constant (e.g., 1.6) to obtain an accurate mass-to-volume ratio of the actual measure of microplastics in target body of water.

In general, the fluid conduit 9 is designed to enable the flow of sample water from the target body of water. As can be seen in FIGS. 1 through 9, for example, the fluid conduit 9 can be a pipe large enough to enable the sample water to flow at a predetermined volumetric flow rate. The flow of the sample water can be enabled using different methods. Passive methods such as water currents of the body of water can be utilized. Other passive methods such as the use of gravity can be implemented to enable the flow of sample water through the fluid conduit 9. Alternatively, active methods such as the use of pumps can be implemented to ensure a predetermined volumetric flow rate is implemented for the flow of the sample water.

As can be seen in FIGS. 1 through 9, the present invention may further comprise at least one fluid pump 12 that drives the flow of sample water through the fluid conduit 9. In addition, the fluid conduit 9 may comprise at least one conduit inlet 10 and at least one conduit outlet 11. The at least one conduit inlet 10 enables water from the targe body of water to flow into the fluid conduit 9, while the at least one conduit outlet 11 enables the sample water to exit the fluid conduit 9. In general, the at least one fluid pump 12 can be implemented as follows: the at least one conduit inlet 10, the at least one conduit outlet 11, and the at least one fluid pump 12 are in fluid communication with each other so that the flow of sample of water is driven by the at least one fluid pump 12. For example, the at least one fluid pump 12 can be positioned adjacent to the at least one conduit outlet 11 so that water from the target body of water is suctioned into the fluid conduit 9 through the at least one conduit inlet 10. In addition, the electronics housing 1 is positioned offset to the at least one fluid pump 12 so that once the sample water flows into the fluid conduit 9, the sample water is exposed to the emitted UV light and the radiated microplastics emit light as a result. Then, the sample water flows out of the fluid conduit 9 to maintain a constant analysis of the targe body of water. Furthermore, the operation of the at least one fluid pump 12 may be also monitored by the controller 7 and the electrical power necessary for operation can be provided by the power source 8. Alternatively, the at least one fluid pump 12 may operate independent from the rest of the system with a separate control system and independent power source.

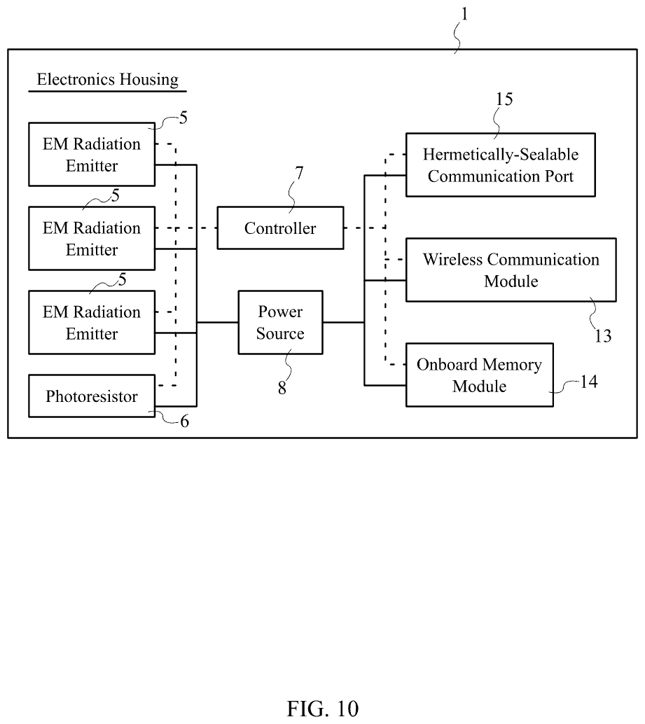

As previously discussed, the controller 7 and the power source 8 can be implemented within the electronics housing 1 to make the system of the present invention a self-contained structure, as can be seen in FIG. 10. In this embodiment, the power source 8 can be a portable power source 8 that fits within the electronics housing 1. The controller 7 can also be a microcontroller 7 that fits inside the electronics housing 1. This enables the controller 7 and the portable power source 8 to be mounted within the electronics housing 1. Thus, different units of the system can be deployed in separate units of the fluid conduit 9 to analyze different water samples from the same body of water or from different bodies of water.

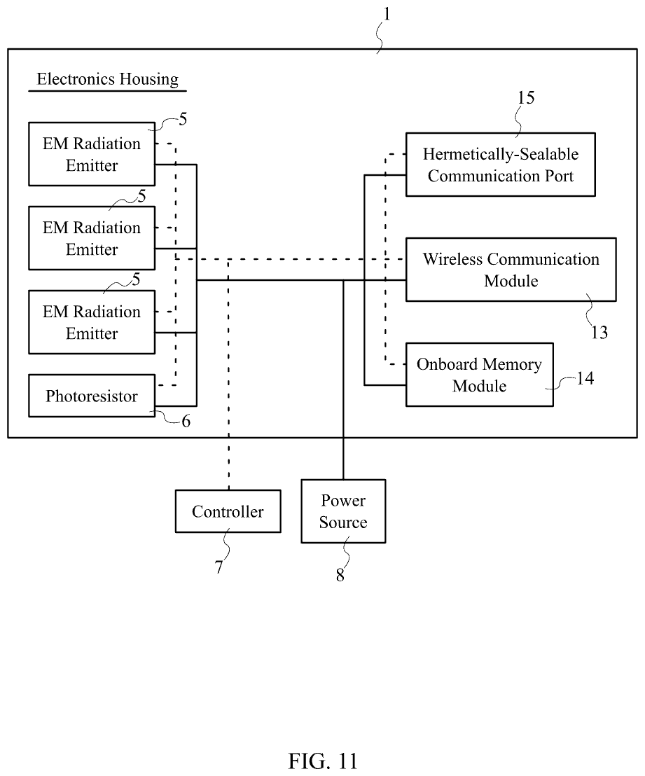

In some embodiments, the present invention may further comprise a wireless communication module 13 that enables the wireless transmission of sensor data from the system to an external computing device, as can be seen in FIGS. 10 and 11. This allows users to monitor real-time data as the target body of water is being analyzed. In general, the wireless communication module 13 can be mounted within the electronics housing 1 to protect the wireless communication module 13 from water damage. The wireless communication module 13 is electronically connected to the controller 7 to relay the generated analysis data from the controller 7 to the external computing device. Further, the wireless communication module 13 is electrically connected to the power source 8 to receive the electrical power necessary for operation.

If the system is not equipped with a wireless communication module 13, the present invention may further comprise an onboard memory module 14 that allows the local storage of the generated analysis data, as can be seen in FIGS. 10 and 11. The onboard memory module 14 allows the safe storage of the generated analysis data until the data can be safely retrieved. The onboard memory module 14 is mounted within the electronics housing 1 to prevent water damage to the onboard memory module 14 during the analysis of the target body of water. The onboard memory module 14 is also electronically connected to the controller 7 so that the generated analysis data can be relayed to the onboard memory module 14. Further, the onboard memory module 14 is electrically connected to the power source 8 to receive the electrical power necessary for the operation of the onboard memory module 14.

Further, when the analysis data is locally stored, the present invention may further comprise a hermetically-sealable communication port 15 that allows the temporary connection of portable data storage device to the system to retrieve the stored analysis data, as can be seen in FIGS. 10 and 11. For example, the hermetically-sealable communication port 15 can be designed to accommodate Secure Digital (SD) cards or other appropriate portable data storage devices. So, the hermetically-sealable communication port 15 can be integrated into the electronics housing 1 in such a way that the hermetically-sealable communication port 15 is protected from water damage. In addition, the hermetically-sealable communication port 15 is electronically connected to the controller 7 to relay the locally stored analysis data to the connected portable data storage device. Furthermore, the hermetically-sealable communication port 15 is electrically connected to the power source 8 to enable the operation of the hermetically-sealable communication port 15.

Although the invention has been explained in relation to its preferred embodiment, it is to be understood that many other possible modifications and variations can be made without departing from the spirit and scope of the invention.

What is claimed is:

1. A system for measuring microplastics in an aquatic environment, the system comprising:
an electronics housing;
a plurality of electromagnetic radiation emitters;
at least one photoresistor;
a controller;
a power source;
a fluid conduit;
the electronics housing comprising an opaque housing portion and a transparent housing portion;
the opaque housing portion being perimetrically positioned around the transparent housing portion;
the opaque housing portion being mounted within the fluid conduit;
the transparent housing portion being oriented towards an interior of the fluid conduit;
the plurality of electromagnetic radiation emitters and the at least one photoresistor being mounted within the electronics housing;
the plurality of electromagnetic radiation emitters and the at least one photoresistor being positioned adjacent to the transparent housing portion;
the plurality of electromagnetic radiation emitters and the at least one photoresistor being electronically connected to the controller; and
the plurality of light sources, the at least one photoresistor, and the controller being electrically connected to the power source.

2. The system as claimed in claim 1, wherein the plurality of electromagnetic radiation emitters is a plurality of ultraviolet (UV) light emitting diodes (LEDs).

3. The system as claimed in claim 1, wherein the fluid conduit is made from an opaque material.

4. The system as claimed in claim 1 further comprising:
the electronics housing further comprising a housing slot;
the transparent housing portion being a transparent slot cover;
the housing slot traversing through the opaque housing portion and into the electronics housing; and
the transparent slot cover being hermetically mounted into the housing slot.

5. The system as claimed in claim 1 further comprising:
the plurality of electromagnetic radiation emitters being distributed around the at least one photoresistor.

6. The system as claimed in claim 1 further comprising:
at least one fluid pump;
the fluid conduit comprising at least one conduit inlet and at least one conduit outlet;
the at least one conduit inlet, the at least one conduit outlet, and the at least one fluid pump being in fluid communication with each other; and
the electronics housing being positioned offset to the at least one fluid pump.

7. The system as claimed in claim 1 further comprising:
the controller being mounted within the electronics housing.

8. The system as claimed in claim 1 further comprising:
the power source being a portable power source; and
the portable power source being mounted within the electronics housing.

9. The system as claimed in claim 1 further comprising:
a wireless communication module;
the wireless communication module being mounted within the electronics housing;
the wireless communication module being electronically connected to the controller; and
the wireless communication module being electrically connected to the power source.

10. The system as claimed in claim 1 further comprising:
an onboard memory module;
the onboard memory module being mounted within the electronics housing;
the onboard memory module being electronically connected to the controller; and
the onboard memory module being electrically connected to the power source.

11. The system as claimed in claim 1 further comprising:
a hermetically-sealable communication port;
the hermetically-sealable communication port being integrated into the electronics housing;
the hermetically-sealable communication port being electronically connected to the controller; and
the hermetically-sealable communication port being electrically connected to the power source.

12. A system for measuring microplastics in an aquatic environment, the system comprising:
an electronics housing;
a plurality of electromagnetic radiation emitters;
at least one photoresistor;
a controller;
a power source;
a fluid conduit;
at least one fluid pump;
the electronics housing comprising an opaque housing portion and a transparent housing portion;
the fluid conduit comprising at least one conduit inlet and at least one conduit outlet;
the opaque housing portion being perimetrically positioned around the transparent housing portion;
the opaque housing portion being mounted within the fluid conduit;
the transparent housing portion being oriented towards an interior of the fluid conduit;
the plurality of electromagnetic radiation emitters and the at least one photoresistor being mounted within the electronics housing;
the plurality of electromagnetic radiation emitters and the at least one photoresistor being positioned adjacent to the transparent housing portion;
the at least one conduit inlet, the at least one conduit outlet, and the at least one fluid pump being in fluid communication with each other;
the electronics housing being positioned offset to the at least one fluid pump;
the plurality of electromagnetic radiation emitters and the at least one photoresistor being electronically connected to the controller; and
the plurality of light sources, the at least one photoresistor, and the controller being electrically connected to the power source.

13. The system as claimed in claim 12, wherein the plurality of electromagnetic radiation emitters is a plurality of ultraviolet (UV) light emitting diodes (LEDs).

14. The system as claimed in claim 12, wherein the fluid conduit is made from an opaque material.

15. The system as claimed in claim 12 further comprising:
the electronics housing further comprising a housing slot;
the transparent housing portion being a transparent slot cover;
the housing slot traversing through the opaque housing portion and into the electronics housing; and
the transparent slot cover being hermetically mounted into the housing slot.

16. The system as claimed in claim 12 further comprising:
the plurality of electromagnetic radiation emitters being distributed around the at least one photoresistor.

17. The system as claimed in claim 12 further comprising:
the power source being a portable power source;
the controller being mounted within the electronics housing; and
the portable power source being mounted within the electronics housing.

18. The system as claimed in claim 12 further comprising:
a wireless communication module;
the wireless communication module being mounted within the electronics housing;
the wireless communication module being electronically connected to the controller; and
the wireless communication module being electrically connected to the power source.

19. The system as claimed in claim 12 further comprising:
an onboard memory module;
the onboard memory module being mounted within the electronics housing;
the onboard memory module being electronically connected to the controller; and
the onboard memory module being electrically connected to the power source.

20. The system as claimed in claim 12 further comprising:
a hermetically-sealable communication port;
the hermetically-sealable communication port being integrated into the electronics housing;
the hermetically-sealable communication port being electronically connected to the controller; and
the hermetically-sealable communication port being electrically connected to the power source.
